(19) United States
(12) Patent Application Publication
(10) Pub. No.: US 2022/0170859 A1
(43) Pub. Date: Jun. 2, 2022

(54) PARTICLE LABELING REAGENT, PARTICLE DYEING METHOD, AND MICROSCOPIC SYSTEM

(71) Applicant: SONY GROUP CORPORATION, TOKYO (JP)

(72) Inventors: DAISUKE YAMAGUCHI, TOKYO (JP); TAKAHITO SUZUKI, TOKYO (JP); YOSHIO GOTO, TOKYO (JP)

(21) Appl. No.: 17/433,444

(22) PCT Filed: Jan. 9, 2020
(86) PCT No.: PCT/JP2020/000368

§ 371 Date: Aug. 24, 2021

(30) Foreign Application Priority Data
Mar. 6, 2019 (JP) 2019-040833

(51) Int. Cl. G01N 21/64; G01N 33/58

(52) U.S. Cl. CPC G01N 21/6458; G01N 33/583; G01N 2021/6439; G01N 21/6428; G01N 33/582

(57) ABSTRACT

To provide a particle labeling reagent having low luminescent characteristics and improved water solubility.

Provided is a particle labeling reagent containing a compound represented by the following general formula (I-1) or (I-2).

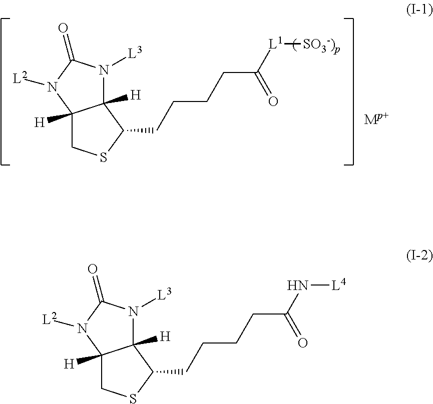

(In the above general formula (I-1), p represents an integer of 1 to 3.
In the above general formula (I-1), M represents a hydrogen atom or a mono- to tri-valent metal atom.
In the above general formula (I-1), L¹ represents a single bond or a (p+1)-valent group.
In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.
In the above general formula (I-2), L⁴ represents a monovalent group.)

### TECHNICAL FIELD

The present technology relates to a particle labeling reagent, a particle dyeing method, and a microscopic system.

### BACKGROUND ART

Dyeing of a particle is one of essential steps for observing the particle. By dyeing a target particle, the target particle can be clearly distinguished from another particle that is not the target particle.

Among methods for dyeing a particle, a method using an avidin-biotin system is known as a representative method, and various study results have been reported in recent years for a dyeing method using this system. For example, Non-Patent Document 1 discloses a method for dyeing a particle with biotion-4 fluorescein (hereinafter, referred to as “B4F”) which is a biotin labeling dye having high water solubility and streptavidin which is a fluorescent label. In this method, two-color dyeing is implemented by localizing biotin in a cell membrane using light fading of B4F and then using streptavidins having different labeling dyes.

As another method, a method using a caged biotin NHS (N-hydroxysuccinimide) ester is also known (Non-Patent Document 2). In this method, cell dyeing is performed with a caged biotin NHS ester, and uncaging is performed by light irradiation. As a result, streptavidin can be adsorbed, and dyeing can be thereby performed.

### CITATION LIST

### Non-Patent Document

Non-Patent Document 1: Binan, L. et al., Nat. Commun., 7, 11636, 2016
Non-Patent Document 2: Terai, T. et al., Chem. Biol., 18, 1261, 2011

### SUMMARY OF THE INVENTION

### Problems to be Solved by the Invention

However, in the method using B4F, when labeling dyes having overlapping excitation or emission wavelengths are selected, the excitation light or emission light overlaps with fluorescence of B4F, and a problem that background noise increases occurs. Furthermore, in the method using a caged biotin NHS ester, a problem that an organic solvent needs to be used occurs because the ester has low water solubility.

Under such circumstances, a main object of the present technology is to provide a particle labeling reagent having low luminescent characteristics and improved water solubility.

### Solutions to Problems

First, the present technology provides a particle labeling reagent containing a compound represented by the following general formula (I-1) or (I-2).

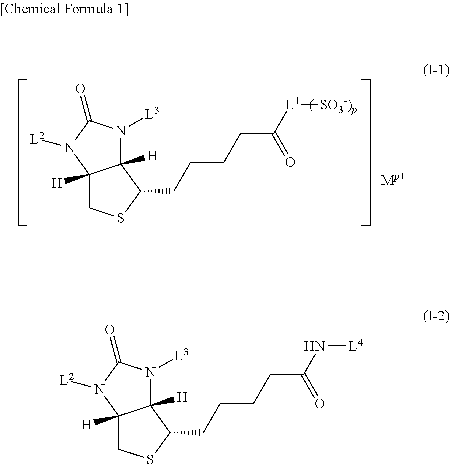

(In the above general formula (I-1), p represents an integer of 1 to 3.

In the above general formula (I-1), M represents a hydrogen atom or a mono- to tri-valent metal atom.

In the above general formula (I-1), L¹ represents a single bond or a (p+1)-valent group.

In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.

In the above general formula (I-2), L⁴ represents a monovalent group.)

In the particle labeling reagent according to the present technology, L² and/or L³ in the general formulas (I-1) and (I-2) can be a monovalent group containing a 2-nitrobenzyl derivative. In this case, the monovalent group containing a 2-nitrobenzyl derivative can be a monovalent group represented by any one of the following general formulas (II-1) to (II-3).

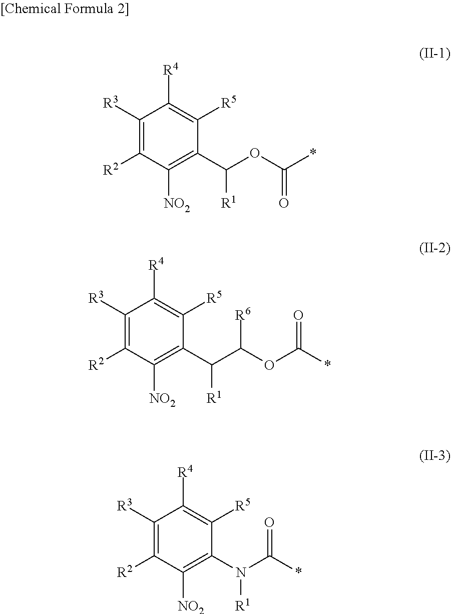

(In the above general formulas (II-1) to (II-3), R¹ and R⁶ each represent a hydrogen atom or a monovalent group. R¹ and R⁶ may be the same or different.

In the above general formulas (II-1) to (II-3), R², R³, R⁴, and R⁵ each independently represent a hydrogen atom or a monovalent group, or represent a ring structure formed by binding R², R³, R⁴, and R⁵ to each other. R², R³, R⁴, and R⁵ may be the same or different.

In the above general formulas (II-1) to (II-3), * represents a bond.)

Furthermore, in this case, any one or more of the group consisting of R², R³, R⁴, and R⁵ in the general formulas (II-1) to (II-3) can each represent a monovalent group containing a polyethylene glycol chain.

In the particle labeling reagent according to the present technology, L¹ in the general formula (I-1) can represent a (p+1)-valent group containing a succinimide ring.

Furthermore, in the particle labeling reagent according to the present technology, L¹ in the general formula (I-1) can represent a (p+1)-valent group containing a polyethylene glycol chain.

Moreover, in the particle labeling reagent according to the present technology, L⁴ in the general formula (I-2) can represent a monovalent lipid-soluble functional group.

In addition, in the particle labeling reagent according to the present technology, L⁴ in the general formula (I-2) can represent a monovalent group containing a polyethylene glycol chain.

Furthermore, in the particle labeling reagent according to the present technology, L⁴ in the general formula (I-2) can represent a monovalent cationic functional group.

Furthermore, the present technology also provides a particle dyeing method including: a primary labeling step of dyeing a target particle with a particle labeling reagent containing a compound represented by the above general formula (I-1) or (I-2) and irradiating the dyed target particle with light; and a secondary labeling step of dyeing the target particle that has been subjected to the primary labeling step with a dye-labeled biotin-binding protein.

In the particle dyeing method according to the present technology, the primary labeling step can further include a binding enabling step in which the photodegradable protecting group is degraded by light irradiation and biotin becomes capable of binding to a biotin-binding protein.

Furthermore, in the particle dyeing method according to the present technology, by repeatedly performing the primary labeling step and the secondary labeling step, biotin-binding proteins in the different secondary labeling steps can be labeled with different dyes.

Moreover, the present technology also provides a microscopic system including: a particle capturing unit that captures a target particle in a well in a particle capturing region; an image acquiring unit that acquires an image of the captured target particle; and an analysis unit that analyzes the image of the target particle acquired by the image acquiring unit, in which the target particle analyzed by the analysis unit is dyed with a particle labeling reagent containing a compound represented by the above general formula (I-1) or (I-2).

The microscopic system according to the present technology can be a microscopic system further including a light irradiation unit that emits light, in which the dyed target particle is primarily labeled by being irradiated with light by the light irradiation unit, and the primarily labeled target particle becomes capable of binding to a dye-labeled biotin-binding protein.

Furthermore, the microscopic system according to the present technology can be a microscopic system further including a particle extracting unit that extracts a target particle, in which the particle extracting unit extracts the target particle binding to the dye-labeled biotin-binding protein.

### BRIEF DESCRIPTION OF DRAWINGS

FIG. 1 is a diagram illustrating an example of site-specific cell labeling using a caged biotin and a fluorescently labeled streptavidin.

FIG. 2 is a diagram illustrating an example of site-specific multicolor dyeing using many types of fluorescently labeled streptavidins.

FIG. 3 is a block diagram of a microscopic system 100 according to the present technology.

FIG. 4 is a diagram illustrating a synthesis scheme of a MeNPOC-biotin-sulfo-NHS ester sodium salt.

FIG. 5 is a diagram illustrating a ¹H-NMR chart of biotin-OMe (2).

FIG. 6 is a diagram illustrating a TOF MS analysis result of biotin-OMe (2).

FIG. 7 is a diagram illustrating a ¹H-NMR chart of MeNPOC-ONP (4).

FIG. 8 is a diagram illustrating a ¹H-NMR chart of a collected sample (MeNPOC-biotin-OMe (5) and contaminants).

FIG. 9 is a diagram illustrating a TOF MS analysis result of a collected sample (MeNPOC-biotin-OMe (5) and contaminants).

FIG. 10 is a diagram illustrating a ¹H-NMR chart of MeNPOC-biotin-OH (6).

FIG. 11 is a diagram illustrating a MALDI-TOF MS analysis result of MeNPOC-biotin-OH (6).

FIG. 12 is a diagram illustrating a ¹H-NMR chart of MeNPOC-biotin-sulfo-NHS ester Na (7).

FIG. 13 is a diagram illustrating a MALDI-TOF MS analysis result of MeNPOC-biotin-sulfo-NHS ester Na (7).

FIG. 14 is a diagram illustrating a change in ¹H-NMR spectrum due to UV irradiation.

FIG. 15 is a diagram illustrating a principle of biotin quantification by a HABA method.

FIG. 16 is a diagram illustrating estimation of a D-biotin concentration by a HABA method.

FIG. 17 is a diagram comparing an adsorption amount of an Alexa Fluor-488 labeled streptavidin between a sample with cell dyeing with MeNPOC-biotin-sulfo-NHS ester Na and a sample without cell dyeing with MeNPOC-biotin-sulfo-NHS ester Na, and between a sample with UV irradiation and a sample without UV irradiation.

FIG. 18 is a flowchart illustrating an example of a particle dyeing method according to the present technology.

### MODE FOR CARRYING OUT THE INVENTION

Hereinafter, a preferred embodiment for carrying out the present technology will be described with reference to the drawings.

The embodiment described below exemplifies representative embodiments of the present technology, and the scope of the present technology is not narrowly interpreted by the embodiment. Note that the description will be made in the following order.

1. Particle labeling reagent

(1) Compound represented by the above general formula (I-1)

(2) Compound represented by the above general formula (I-2)

2. Particle dyeing method

(1) Primary labeling step

(2) Secondary labeling step

(3) Other step

3. Microscopic system 100

(1) Particle capturing unit 1

(2) Image acquiring unit 2

(3) Analysis unit 3

(4) Light irradiation unit 4

(5) Particle extracting unit 5

(5-1) Association unit

(5-2) Particle discharging unit

(5-3) Distinguishment information acquiring unit

(5-4) Confirmation unit

(5-5) Others

(6) Observation unit 6

(7) Control unit 7

(8) Storage unit 8

(9) Display unit 9

1. Particle Labeling Reagent

A particle labeling reagent according to the present technology contains a compound represented by the above general formula (I-1) or (I-2).

Examples of a particle to be labeled using the particle labeling reagent according to the present technology include, but are not limited to, a biological microparticle such as a cell, a microorganism, a solid component derived from a living body, or a liposome, and a synthetic particle such as a latex bead, a gel bead, a magnetic bead, or a quantum dot. Furthermore, the cell may include an animal cell and a plant cell. Examples of the animal cell may include a tumor cell and a blood cell. The microorganism may include bacteria such as Escherichia coli and fungi such as yeast. Examples of the solid component derived from a living body may include a solid crystal generated in a living body. The synthetic particle may be, for example, a particle containing an organic or inorganic polymer material or a metal. The organic polymer material may include polystyrene, styrene-divinylbenzene, polymethyl methacrylate, and the like. The inorganic polymer material may include glass, silica, a magnetic material, and the like. The metal may include gold colloid, aluminum, and the like. Furthermore, the particle may be, for example, a conjugate of a plurality of particles such as two or three particles. Moreover, the particle does not need to be fixed onto a two-dimensional plane, and may be floating. Note that here, a particle to be dyed is referred to as a “target particle”.

The particle labeling reagent according to the present technology has low luminescent characteristics, and therefore does not cause a problem that background noise increases due to overlapping with fluorescence of the reagent. Therefore, the particle labeling reagent according to the present technology does not narrow a use wavelength range of a next stage labeling dye. Furthermore, the particle labeling reagent according to the present technology has high water solubility and does not require an organic solvent for dissolution in water, and therefore can also prevent damage to a particle and the like.

(1) Compound Represented by the Above General Formula (I)

In the above general formula (I-1), p represents an integer of 1 to 3, but p is preferably 1 or 2 and more preferably 1.

In the above general formula (I-1), M represents a hydrogen atom or a mono- to tri-valent metal atom. Examples of the metal atom include: an alkali metal such as sodium or potassium; an alkaline earth metal such as magnesium, calcium, strontium, or barium; aluminum; and iron. In the present technology, the metal atom is preferably an alkali metal, and among these, sodium and potassium are preferable in the present technology.

In the above general formula (I-1), L¹ represents a single bond or a (p+1) group, but is preferably a (p+1)-valent group, more preferably a divalent or trivalent group, and still more preferably a divalent group. Note that in the present technology, the “single bond” means one that directly binds substituents to be linked to each other.

L¹ is preferably a (p+1)-valent group having amine reactivity. The (p+1)-valent group having amine reactivity may have acquired amine reactivity including a carbonyl group binding to L¹. Specific examples thereof include (p+1)-valent groups including isothiocyanate, isocyanate, acyl azide, NHS (N-hydroxysuccinimide) ester, sulfonyl chloride, aldehyde, glyoxal, epoxide, oxirane, carbonate, aryl halide, imide ester, carbodiimide, anhydride, and fluoroester.

In the present technology, among these, the (p+1)-valent group having amine reactivity is preferably a (p+1)-valent group containing a succinimide ring. Specific examples thereof include groups represented by the following general formulas (III-1) to (III-3). Note that in the following general formulas (III-1) to (III-3), * represents a binding site to a carbonyl carbon in the above general formula (I-1), and ** represents a binding site to a sulfur atom in the above general formula (I-1).

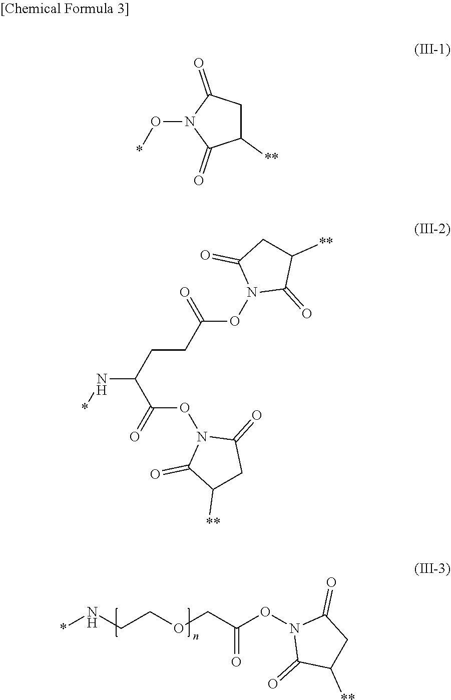

In the above general formula (III-3), n represents the number of repeating units of a polyethylene glycol chain, and is an integer of 1 or more. In the present technology, as illustrated in the above general formula (III-3), L¹ can represent a (p+1)-valent group containing a polyethylene glycol chain. n is preferably 1 to 1000, more preferably 2 to 500, and still more preferably 3 to 230. This makes it possible to improve stability in an aqueous solution.

In the above general formulas (I-1) and (I-2), L² and L³ each independently represent a photodegradable protecting group, and L² and L³ may be the same or different. Provided that at least one of L² and L³ represents a photodegradable protecting group. As described above, by introducing the photodegradable protecting group into the compound, the activity of the compound can be turned on (or off) by light irradiation.

The photodegradable protecting group is preferably a monovalent group containing a 2-nitrobenzyl derivative. The monovalent group containing a 2-nitrobenzyl derivative can be, for example, a monovalent group represented by any one of the above general formulas (II-1) to (II-3). Note that in the above general formulas (II-1) to (II-3), * represents a bond.

In the general formulas (II-1) to (II-3), R¹ and R⁶ each represent a hydrogen atom or a monovalent group. R¹ and R⁶ may be the same or different. Examples of the monovalent group include a monovalent chain hydrocarbon group (alkyl, alkenyl, alkynyl, and the like), a monovalent alicyclic hydrocarbon group (cycloalkyl, cycloalkenyl, cycloalkynyl, and the like), a monovalent aromatic hydrocarbon group (aryl such as phenyl or naphthyl, and the like), a monovalent aromatic heterocyclic group (pyrenyl, pyrrolyl, furanyl, thiophenyl, pyridinyl, pyridazinyl, pyrimidinyl, pyrazinyl, triazinyl, and the like), a monovalent nonaromatic heterocyclic group (oxiranyl, aziridinyl, azetidinyl, oxetanyl, thietanyl, pyrrolidinyl, dihydrofuranyl, tetrahydrofuranyl, and the like), and a combination thereof.

R¹ and R⁶ may each have a substituent. Examples of the substituent include a halogen atom (fluorine atom, chlorine atom, bromine atom, iodine atom, and the like), carboxy, sulfo, cyano, nitro, mercapto, oxo, guanidino, hydroxy, alkyl, alkoxy, alkylcarbonyl, alkyloxycarbonyl, alkylcarbonyloxy, amino (amino and the like) which may be subjected to mono- or di-substitution with alkyl, and amino-carbonyl (amide and the like) which may be subjected to mono- or di-substitution with alkyl.

R¹ or R⁶ is preferably a hydrogen atom or an alkyl group having 1 to 5 carbon atoms, and more preferably a hydrogen atom or an alkyl group having 1 to 3 carbon atoms. Examples of the alkyl group having 1 to 5 carbon atoms include: a linear alkyl group such as a methyl group, an ethyl group, a n-propyl group, a n-butyl group, or a n-pentyl group; and a branched alkyl group such as an isopropyl group, an isobutyl group, a sec-butyl group, a t-butyl group, or an isoamyl group.

In the above general formulas (II-1) to (II-3), R², R³, R⁴, and R⁵ each independently represent a hydrogen atom or a monovalent group, or represent a ring structure formed by binding R², R³, R⁴, and R⁵ to each other. R², R³, R⁴, and R⁵ may be the same or different. The monovalent group is similar to that described in the description of R¹ and R⁶, and therefore description thereof is omitted here.

The ring structure can be, for example, a ring structure having 3 to 10 ring members. Examples of the ring structure having 3 to 10 ring members include: a cycloalkane structure such as a cyclopropane structure, a cyclobutane structure, a cyclopentane structure, a cyclohexane structure, a norbornane structure, or an adamantane structure; an oxacycloalkane structure such as an oxacyclobutane structure, an oxacyclopentane structure, an oxacyclohexane structure, an oxanorbornane structure, or an oxaadamantane structure; and an aromatic ring structure such as a benzene ring structure or a naphthalene ring structure. The substituent may bind to each of these ring structures. Furthermore, —O—, —COO—, —SO₂O—, —NR^{a}SO₂, —NR^{a}CO—, and the like may be contained between carbon and carbon of the ring structure. Here, R^{a} can be, for example, a hydrogen atom or a hydrocarbon group having 1 to 10 carbon atoms.

Any one or more of the group consisting of R², R³, R⁴, and R⁵ are each preferably a substituted or unsubstituted alkoxy group, or any two or more of the group consisting of R², R³, R⁴, and R⁵ preferably form a ring structure by binding substituted or unsubstituted alkoxy groups to each other.

Any one group selected from the group consisting of a 2-nitrobenzyl group, a 4,5-dimethoxy-2-nitrobenzyl group, a 2-nitrophenethyl group, an α-carboxy-2 nitrobenzyl group, an α-methyl-4,5-dimethoxy-2-nitrobenzyl group, and an α-methyl-6-nitro-piperonyl group, or a group represented by the following general formula (IV) preferably binds to a binding site to a carbon atom binding to R¹ in the above general formula (II-1) or (II-2) or a binding site to a nitrogen atom binding to R¹ in the above general formula (II-3). Note that in the following general formula (IV), * represents a binding site to a carbon atom binding to R¹ in the above general formula (II-1) or (II-2) or a binding site to a nitrogen atom binding to R¹ in the above general formula (II-3).

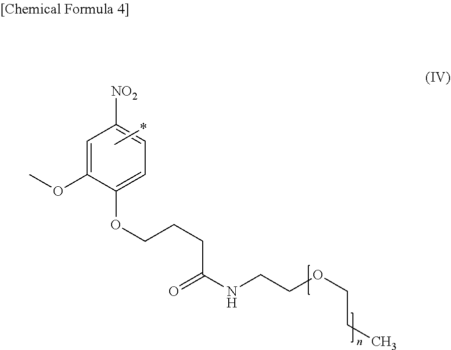

In the above general formula (IV), n represents the number of repeating units of a polyethylene glycol chain, and is an integer of 1 or more. In the present technology, as illustrated in the above general formula (IV), any one or more of the group consisting of R², R³, R⁴, and R⁵ in the above general formulas (II-1) to (II-3) can each represent a monovalent group containing a polyethylene glycol chain. n is preferably 1 to 1000, more preferably 2 to 500, and still more preferably 3 to 230. This makes it possible to improve stability in an aqueous solution.

Specific examples of a compound represented by the above general formula (I-1) are illustrated in the following chemical formulas (I-1-1) to (I-1-4).

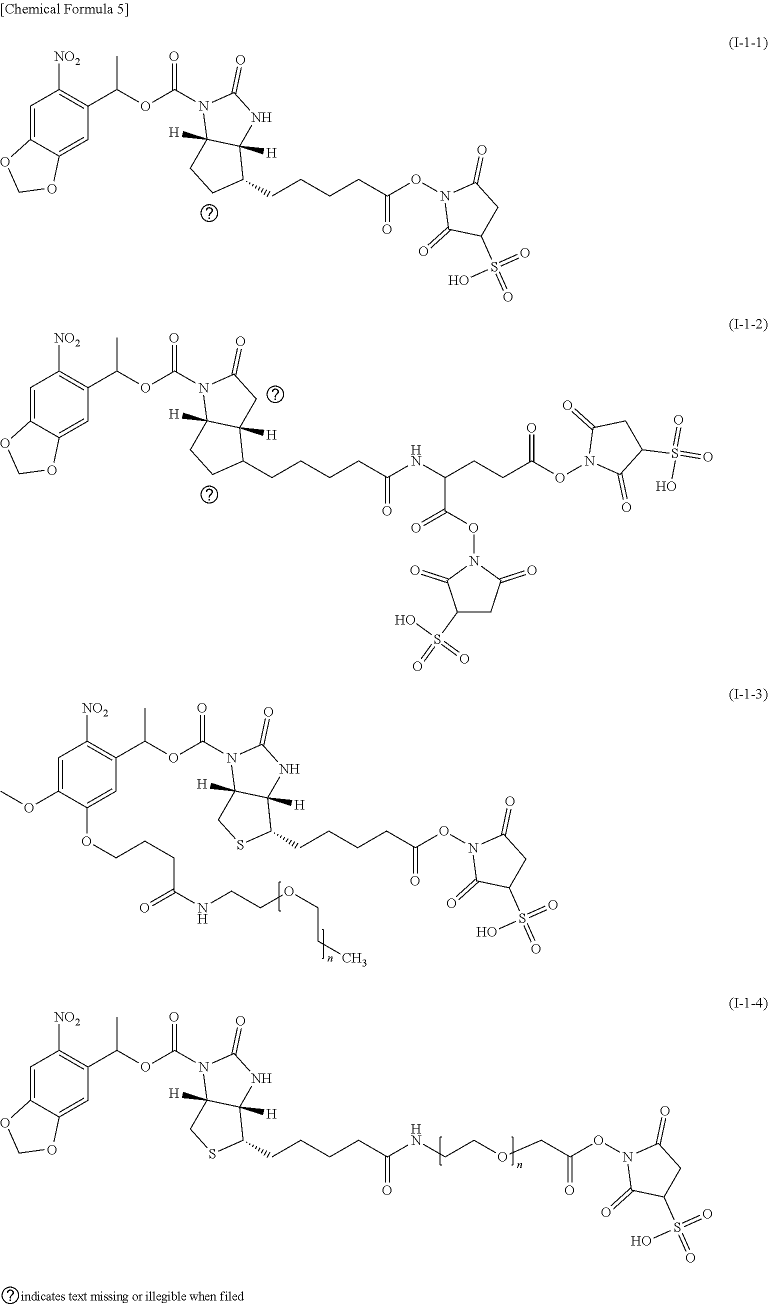

(2) Compound Represented by the Above General Formula (I-2)

A compound represented by the above general formula (I-2) has an amide bond in addition to the above characteristics, and therefore is stable in an aqueous solution.

L² and L³ in the above general formula (I-2) are similar to those described above, and therefore description thereof is omitted here.

In the above general formula (I-2), L⁴ represents a monovalent group. The monovalent group is preferably a monovalent lipid-soluble functional group or a monovalent cationic functional group. Examples of the monovalent lipid-soluble functional group include a group having a phospholipid, a group having a steroid skeleton, and a group having a saturated or unsaturated linear or branched hydrocarbon chain.

Examples of the group having a phospholipid include groups containing DLPE: 1,2-dilauroyl-sn-glycero-3-phosphoethanolamine, DMPE: 1,2-dimyristoyl-sn-glycero-3-phosphoethanolamine, DPPE: 1,2-dipalmitoyl-sn-glycero-3-phosphoethanolamine, DSPE: 1,2-distearoyl-sn-glycero-3-phosphoethanolamine, DOPE: 1,2-dioleoyl-sn-glycero-3-phosphoethanolamine, DLoPE: 1,2-dilinoleoyl-sn-glycero-3-phosphoethanolamine, DEPE: 1,2-dierucoyl-sn-glycero-3-phosphoethanolamine, and POPE: 1-palmitoyl-2-oleoyl-sn-glycero-3-phosphoethanolamine.

Examples of the group having a steroid skeleton include a sterol residue. Examples of the sterol residue include a group obtained by removing a hydrogen atom from a hydroxy group of a sterol such as cholesterol, phytosterol (α-, or γ-sitosterol, stigmasterol, campesterol, spinasterol, brassicasterol, and the like), estriol, estrone, aldosterone, corticosterone, cortisone, cholic acid, glycocholic acid, cymarin, lumisterol, cholestanol (dihydrocholesterol), β-sitostanol (dihydrositosterol), or spinastanol (dihydrospinasterol).

Furthermore, in the present technology, L⁴ can represent a monovalent group containing a polyethylene glycol chain. The number of repeating units of the polyethylene glycose chain is preferably 1 to 1000, more preferably 2 to 500, and still more preferably 3 to 230. This makes it possible to improve stability in an aqueous solution.

Examples of the monovalent cationic functional group include spermidine, spermine, linear or branched polyethyleneimine, an aminoethyl acrylic polymer (POLYMENT (registered trademark); manufactured by Nippon Shokubai Co., Ltd.), and a group containing an alkyl ester of a basic amino acid (lysine, arginine, ornithine, citrulline, and the like). Furthermore, in the present technology, in a case where L⁴ contains these functional groups, the compound represented by the above general formula (I-2) may be a salt, and can be, for example, an inorganic salt such as a hydrochloride, a bromate, a sulfate, or a phosphate, or an organic acid salt such as a glycolate, an acetate, a lactate, a succinate, a tartrate, a citrate, or an acidic amino acid salt.

Specifically, L⁴ can be, for example, a monovalent group represented by any one of the following general formulas (V-1) to (V-9). Note that in the following general formulas (V-1) to (V-9), * represents a binding site to a nitrogen atom in the above general formula (I-2).

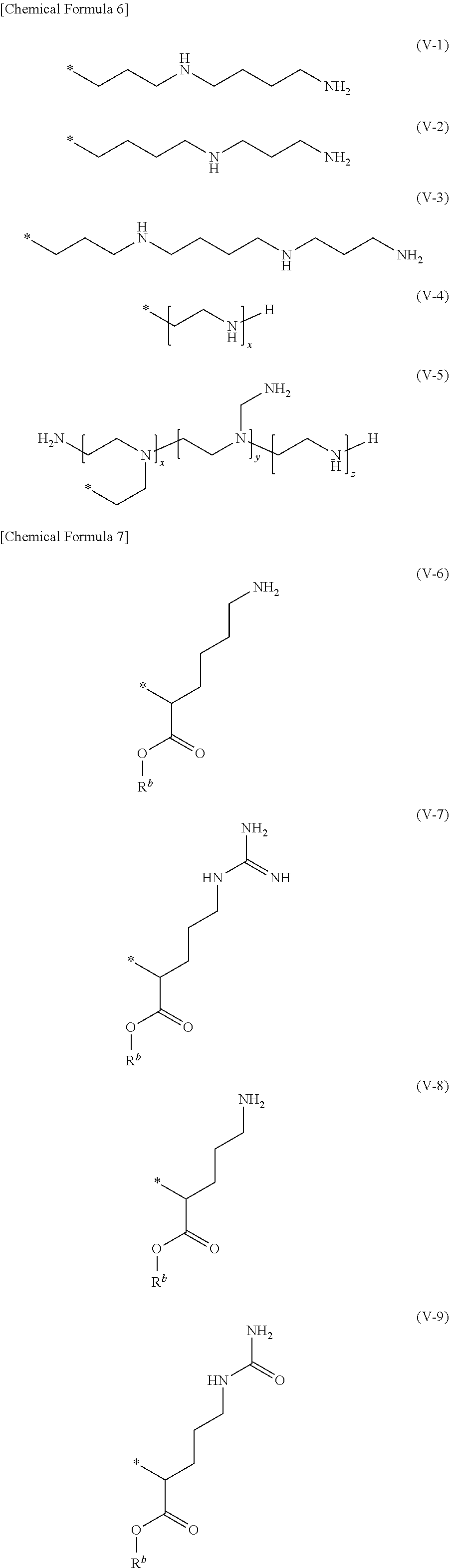

In the above general formula (V-5), x, y, and z each represent an arbitrary integer of 1 or more. Furthermore, in the above general formulas (V-6) to (V-9), R^{b} represents a monovalent group. The monovalent group is similar to that described in the description of R¹ and R⁶, and therefore description thereof is omitted here. R^{b} is preferably an alkyl group having 1 to 5 carbon atoms, and more preferably an alkyl group having 1 to 3 carbon atoms.

Specific examples of the compound represented by the above general formula (I-2) are illustrated in the following chemical formulas (I-2-1) to (I-2-14).

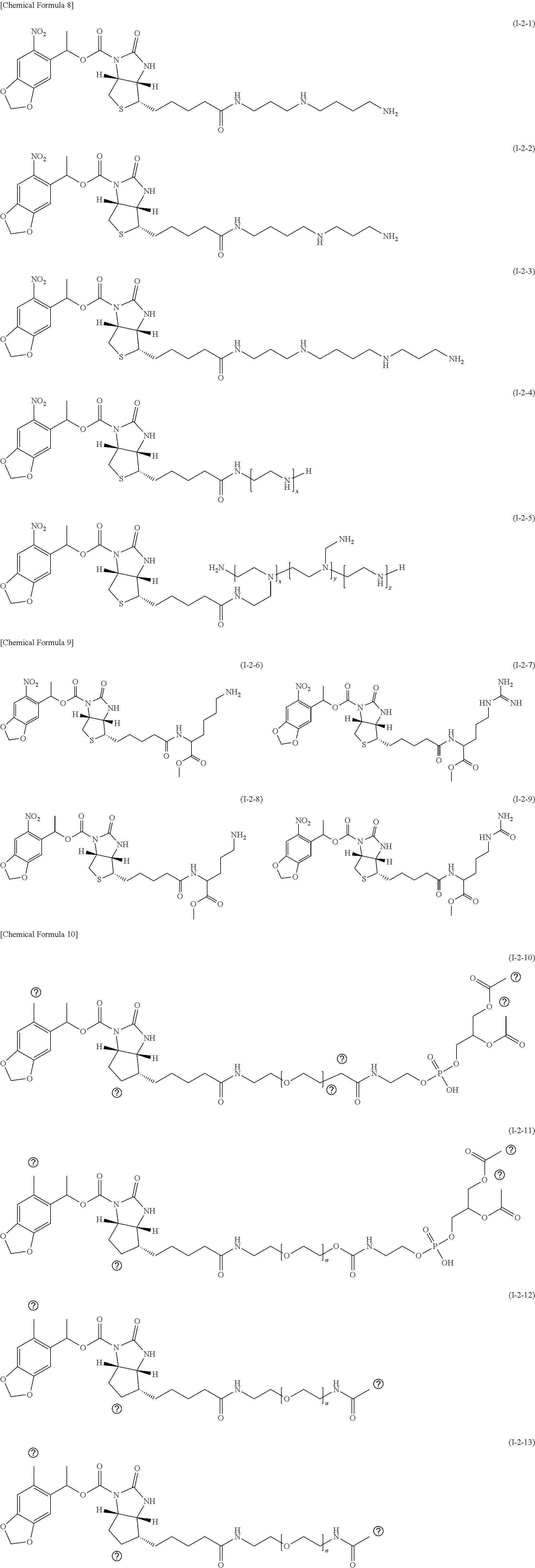

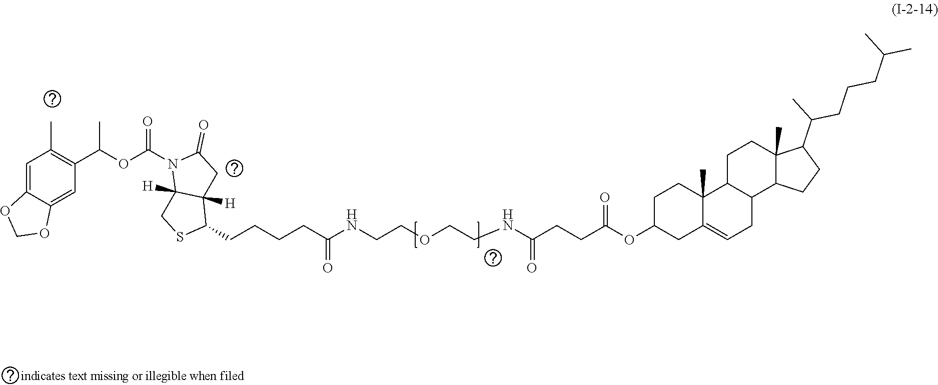

In the above general formula (I-2-5), x, y, and z each represent an arbitrary integer of 1 or more. In the above general formulas (I-2-10) to (I-2-14), n represents the number of repeating units of a polyethylene glycol chain, and is an integer of 1 or more. Furthermore, m represents an integer of 1 or more.

2. Particle Dyeing Method

The particle dyeing method according to the present technology includes a primary labeling step and a secondary labeling step. Furthermore, the particle dyeing method according to the present technology may include another step as necessary. Hereinafter, each step will be described in detail.

(1) Primary Labeling Step

The primary labeling step is a step of dyeing a target particle with a particle labeling reagent containing a compound represented by the above general formula (I-1) or (I-2), and irradiating the dyed target particle with light. The particle labeling reagent is similar to that described above, and therefore description thereof is omitted here.

As the light (uncaging light) with which the dyed target particle is irradiated, for example, laser light having an appropriate wavelength and intensity is used in order to degrade the photodegradable protecting group. As a laser light source, for example, a light source similar to laser light included in a conventionally known flow cytometer can be used, and a mercury lamp, a xenon lamp, or various laser light sources (solid state laser, gas laser, semiconductor laser, and the like) can be used. Furthermore, the wavelength of the laser light is preferably 300 to 450 nm.

In the present technology, the primary labeling step may further include a binding enabling step in which the photodegradable protecting group is degraded by light irradiation and biotin becomes capable of binding to a biotin-binding protein. Examples of the biotin-binding protein include avidin, streptavidin, NeutrAvidin, and an avidin-like protein. In the present technology, these biotin-binding proteins are dye-labeled as described later.

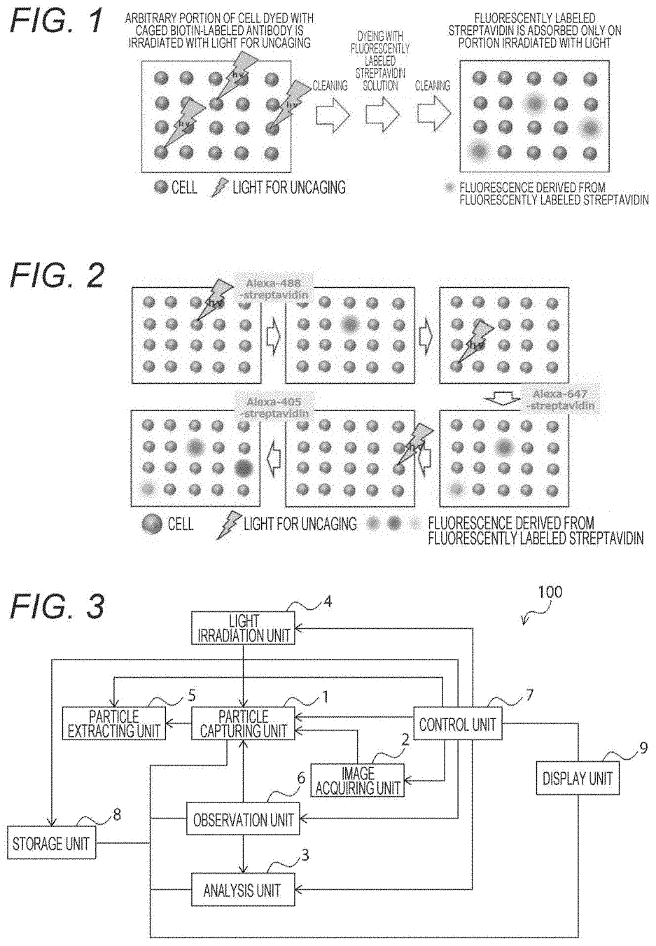

In the binding enabling step, as illustrated in FIG. 1, only an arbitrary region (for example, a region for each well) is irradiated with light to perform uncaging only on some particles, and affinity with a biotin-binding protein can be locally improved. As a result, the dye-labeled biotin-binding protein is adsorbed only on a particle present in a target range, and site-specific particle labeling can be performed. Furthermore, in a case of performing a particle extracting step described later, a particle can also be optically selected.

(2) Secondary Labeling Step

The secondary labeling step is a step of dyeing a target particle that has been subjected to the primary labeling step with a dye-labeled biotin-binding protein.

A dye that can be used is a dye having luminescent characteristics. Specific examples thereof include CF Dyes (Biotium, Inc.), DY (Dyomics GmbH), DyLight Fluor (Dyomics GmbH), Alexa Fluor (Thermo Fisher Scientific), BD Horizon Brilliant (Sirigen, BD Biosciences), Cy (GE Healthcare), HyLyte Fluor, CyLyte Fluor (Anaspec, Inc), ADS (American Dye Source, Inc.), ATTO (ATTO-TEC GmbH), IRDye (Li-COR Biosciences), Pacific Blue, Pacific Green, Pacific Orange (Thermo Fisher Scientific), Texas Red (Thermo Fisher Scientific), eFluor (Thermo Fisher Scientific), Fire™ (BioLegend), NorthernLights (NorthernLights), MFP (MoBiTec), Tide Fluor (AAT Bioquest, Inc.), CAL Fluor (LGC Biosearch Technologies), Abberior STAR (Abberior), Fluoid (IST), FAM, FITC, TRITC, Rhodamine Green, Rhodamine Green-X, Lucifer Yellow, TET, JOE, Yakima Yellow, VIC, ABY, JUN, ROX, LIZ, NED, PET, HEX, Quasar, Dragonfly orange, TAMRA, FAR-Fuschia, LC Red, PULSAR, WellRed, FAR-Blue, FAR-Green, IRIS-Green One, Spectrum Green, Spectrum Red, ECD, EDANS, aminomethylcoumarin (AMCA), AMCA-X (AdipoGen), BODIPY, BODIPY FL, BODIPY FL-X, Royal Blue, Marina blue, Oyster (Luminartis), Oregon Green, Cascade Blue (Thermo Fisher Scientific), tandem structures thereof, and mixtures thereof.

Furthermore, the dye may be a fluorescent protein. Specific examples thereof include GFP, mCherry, Phycoerythrin (PE), Phycocyanin (PC), Allophycocyanin (APC), and Peridinin-Chlorophyll Protein Complex (PerCP). Moreover, the dye can be a semiconductor quantum dot having a surface covered with the biotin-binding protein. Specific examples thereof include Ag₂S, AgInS₂, CuInS₂, and a quantum dot having a core-shell structure with Ag₂S, AgInS₂, or CuInS₂ as a core and ZnS as an outer shell.

Furthermore, in a case where a particle extracting step described later is performed, as the dye, a dye that can be excited by a wavelength of a light source usually used in a flow cytometer can be used. This makes it possible to collect a particle at high speed.

In the present technology, the biotin-binding protein or the dye may be one to which an enzyme binds, or may be a luminescent substrate of an enzyme.

In the present technology, by repeatedly performing the primary labeling step and the secondary labeling step, as illustrated in FIG. 2, biotin-binding proteins in different secondary labeling steps can be labeled with different dyes. As a result, for example, target particles present in arbitrary different regions can be dyed with different dyes, and distinguishment with a plurality of colors can be performed. Furthermore, in a case of performing a particle extracting step described later, a particle can also be optically selected.

(3) Other Step

The particle dyeing method according to the present technology may include another step as necessary. Specific examples thereof include a particle capturing step, an analysis step, and a particle extracting step. The particle capturing step, the analysis step, and the particle extracting step are similar to methods performed by a particle capturing unit 1, an analysis unit 3, and a particle extracting unit 5 of a microscopic system 100 described later, respectively, and therefore description thereof is omitted here. Moreover, as illustrated in Examples described later, a cleaning step and the like can also be appropriately performed.

3. Microscopic System 100

FIG. 3 is a block diagram of the microscopic system 100 according to the present technology. The microscopic system 100 according to the present technology includes the particle capturing unit 1, the image acquiring unit 2, and the analysis unit 3, and a target particle analyzed by the analysis unit 3 is dyed with a particle labeling reagent containing a compound represented by the above general formula (I-1) or (I-2). Furthermore, the microscopic system 100 according to the present technology may include an irradiation unit 4, the particle extracting unit 5, an observation unit 6, a control unit 7, a storage unit 8, a display unit 9, and the like as necessary. The particle labeling reagent is similar to that described above, and therefore description thereof is omitted here.

(1) Particle Capturing Unit 1

The particle capturing unit 1 captures a target particle in a well in a particle capturing region. The particle capturing unit 1 can be constituted by, for example, a plate-like member including a surface of the well on a particle entrance side and a surface facing the surface. The thickness of the plate-like member can be appropriately set according to the depth of the well, the strength of a material of the plate-like member, and the like.

Examples of the material forming the particle capturing unit 1 include an ultraviolet curable resin, particularly a resin applicable to a 3D stereolithography method. The resin can be obtained by, for example, ultraviolet-curing a resin composition containing one or more selected from a silicone elastomer, an acrylic oligomer, an acrylic monomer, an epoxy-based oligomer, an epoxy-based monomer, and the like. Furthermore, a material generally used in the technical field of a microchannel may be used, and examples of the material include glass (borosilicate glass, quartz glass, and the like), a plastic resin (acrylic resin, cycloolefin polymer, polystyrene, and the like), and a rubber material (PDMS and the like). In a case where the particle capturing unit 1 is constituted by a plurality of members, the plurality of members may be constituted by the same material or may be constituted by a combination of different materials.

In the microscopic system 100 according to the present technology, the particle capturing unit 1 can be replaceable.

The well in the particle capturing region can have a shape capable of capturing one particle (preferably, one cell). For example, the shape of a particle entrance in the well can be formed into a circle, an ellipse, a polygon (triangle, quadrangle, and the like), a pentagon, or a hexagon.

Arrangement of the wells is not particularly limited, and can be freely designed according to the form of the particle capturing unit 1 and a purpose after a particle is captured. For example, the wells can be arranged in one row or in a plurality of rows at predetermined intervals, or can be arranged in a lattice pattern at predetermined intervals. The interval in this case can be appropriately selected according to the number of particles to be applied, the number of particles to be captured, and the like. For example, the interval can be designed to 20 μm to 300 μm, preferably 30 μm to 250 μm, more preferably 40 μm to 200 μm, still more preferably 50 μm to 150 μm. Furthermore, the number of wells is not particularly limited, and can be freely set according to a purpose.

A particle captured in the well is observed, subjected to various reactions, subjected to various measurements, and the like according to a purpose.

(2) Image Acquiring Unit 2

The image acquiring unit 2 acquires an image of the captured target particle. Specifically, the image acquiring unit 2 can image a particle capturing surface via an objective lens and the like. The image acquiring unit 2 includes, for example, an image sensor such as a complementary metal oxide semiconductor (CMOS) or a charge coupled device (CCD). Furthermore, the image acquiring unit 2 can transmit an image to the analysis unit 3 described later.

(3) Analysis Unit 3

The analysis unit 3 analyzes an image of a target particle acquired by the image acquiring unit 2. Furthermore, the analysis unit 3 analyzes a target particle dyed with a particle labeling reagent containing a compound represented by the above general formula (I-1) or (I-2). Specifically, the analysis unit 3 can calculate the feature amount of a particle on the basis of an image transmitted from the image acquiring unit 2, and analyze the form, structure, property, and the like of the particle on the basis of the feature amount.

The analysis unit 3 may be implemented by a personal computer or a CPU, can be stored as a program in a hardware resource including a recording medium (non-volatile memory (USB memory), HDD, CD, and the like) and can be caused to function by the personal computer or the CPU. Furthermore, the analysis unit 3 may be connected to each unit of the microscopic system 100 via a network.

(4) Light Irradiation Unit 4

The microscopic system 100 according to the present technology may include the light irradiation unit 4 as necessary. The light irradiation unit 4 irradiates a target particle with light. As a result, the dyed target particle is primarily labeled, and the primarily labeled target particle becomes capable of binding to a dye-labeled biotin-binding protein. The light with which the dyed target particle is irradiated is similar to that described above, and therefore description thereof is omitted here.

(5) Particle Extracting Unit 5

The microscopic system 100 according to the present technology may include the particle extracting unit 5 as necessary. The particle extracting unit 5 extracts a particle, and extracts the target particle binding to the dye-labeled biotin-binding protein. Furthermore, the particle extracting unit 5 can further select a particle from among the target particles.

The particle extracting unit 5 can include, for example, an association unit, a particle discharging unit, a distinguishment information acquiring unit, and a confirmation unit. Hereinafter, each unit will be described in detail.

(5-1) Association Unit

The association unit associates distinguishment information of a particle captured in a well in a particle capturing region with position information of the well. That is, in the associating unit, a particle having distinguishment information is associated with the position of the well in which the particle is captured. The associated distinguishment information and position information are used in the confirmation unit described later.

The association may be performed mechanically, or may be performed by a user who performs a particle extracting operation using the particle capturing region. In a case where the association is performed mechanically, for example, the association may be performed by a system that performs association. The system can be included in the analysis unit 3 and/or the control unit 7 included in the microscopic system 100 according to the present technology.

The “distinguishment information” as used herein may be information used to identify a certain particle from other particles, or may be information used to identify a particle. The distinguishment or the identification can be performed, for example, on the basis of the type or characteristic of a particle. More specifically, the distinguishment information may be information based on fluorescence, color, charge, magnetic charge, or radical of a particle captured in a well, or information regarding the form or size of a particle captured in a well. A combination of the two or more pieces of information may be used as the distinguishment information.

Furthermore, the “position information” as used herein can be information regarding the position of a well in which a particle is captured. The position information may be, for example, information regarding the position of a well in which a particle is captured in one field of view under microscopic observation, or may be information regarding the position of a particle in one field of view under microscopic observation. The position information may be, for example, information regarding the position of a well in which a particle is captured in a particle capturing region, or may be information regarding the position of a particle in a particle capturing region. The position information may be position information in a coordinate system or position information in a non-coordinate system.

The association unit may perform association using an image acquired by the image acquiring unit 2. Distinguishment information of a particle captured in a well and position information of the well are acquired from the image, and then the distinguishment information and the position information can be associated with each other. The distinguishment information acquired from the image can be, for example, fluorescence, color, size, or shape of the particle.

(5-2) Particle Discharging Unit

The particle discharging unit irradiates a well in which a target particle is captured with laser light or an ultrasonic wave, and generates bubbles in the well by the irradiation with the laser light or the ultrasonic wave, thereby discharging the target particle from the well. Specifically, for example, the particle discharging unit generates bubbles by supplying large energy to water in a short time using a laser (for example, a holmium YAG (Ho:YAG) laser) having an oscillation wavelength near a light absorption wavelength of water. Alternatively, a particle operation device such as a micromanipulator or a micropipette can also be used.

Furthermore, a sheet that seals a well in a particle capturing region may be used as the particle discharging unit. Specifically, after a particle is captured, the well in the particle capturing region is sealed by the sheet. Next, a hole is formed only in a sheet portion covering a well in which a target particle is captured, and a flow in which the target particle is discharged from the well is formed. As a result, the target particle can be discharged from the well while other particles are captured in the well. In order to make it possible to form the hole, the sheet may be constituted by, for example, a light ray (for example, an infrared ray and the like) absorbent material. The hole is formed by irradiating the sheet portion with a light ray.

When the target particle is discharged from the well by the particle discharging unit, a plurality of particles having distinguishment information can be continuously discharged. For example, 2 to 100 particles having distinguishment information, particularly 2 to 50 particles having distinguishment information, more particularly 2 to 30 particles having distinguishment information can be continuously discharged. As a result, the order of the pieces of distinguishment information of the plurality of particles continuously discharged can be confirmed by the confirmation unit described later.

In this case, in the present technology, particles may be discharged such that particles having the same distinguishment information are not continuously discharged. That is, the discharge can be performed such that two particles to be continuously discharged have different types of distinguishment information from each other. For example, a particle having red fluorescence, a particle having blue fluorescence, and then a particle having red fluorescence can be discharged in this order. Such an order of the pieces of distinguishment information of the discharged particles can be compared with, for example, the order of the pieces of distinguishment information acquired by the distinguishment information acquiring unit in the following confirmation. In the present technology, as described above, two or more particles to be continuously discharged in the discharge may have different types of distinguishment information. Alternatively, the discharge may be performed such that particles having the same distinguishment information are continuously discharged. For example, three particles having red fluorescence may be continuously discharged. The fact that particles having the same distinguishment information are continuously discharged can also be compared with the order of the pieces of distinguishment information acquired by the analysis unit 3 in the following confirmation.

Furthermore, a particle discharged from a well may be guided to a channel fluidly connected to a space in which a particle capturing region is disposed by a flow of a fluid formed around the particle capturing region, and may be collected in a container or on a plate for particle collection through the channel. Alternatively, a particle discharged from a well may be moved in a channel connected to a micropipette and collected in a container or on a plate for particle collection connected to the channel, or may be moved into a container or onto a plate for particle collection by a micromanipulator. Moreover, particle analysis may be further performed in the container or on the plate. For example, in a case where the particle is a cell, the cell may be analyzed or cultured in the container or on the plate.

(5-3) Distinguishment Information Acquiring Unit

The distinguishment information acquiring unit acquires distinguishment information of a particle. The distinguishment information acquiring unit acquires information after the discharge. That is, in the present technology, distinguishment information of a particle is acquired by two different units of the association unit and the distinguishment information acquiring unit. In the former, a particle is captured in a well, and in the latter, a particle is outside a well. In this way, by acquiring distinguishment information of a particles captured in a well and distinguishment information of the particle after the particle is discharged from the well, it is possible to confirm whether a target particle has been extracted.

The distinguishment information acquiring unit can acquire distinguishment information of a particle collected in one well by, for example, a microscope and the like. Alternatively, the distinguishment information may be acquired from a particle passing through a channel. For example, fluorescence and/or scattered light generated by irradiating a particle passing through a channel with light can be acquired as the distinguishment information. In order to acquire the distinguishment information in this manner, for example, a light detection technique used in a flow cytometer or a cell sorter can be applied.

(5-4) Confirmation Unit

The confirmation unit confirms whether the particle has been captured in a well having the position information on the basis of the acquired distinguishment information. For example, the confirmation unit can confirm the position of the well in which the particle is captured on the basis of the distinguishment information acquired in the distinguishment information acquiring unit.

The confirmation may be performed mechanically, or may be performed by a user who performs a particle extracting operation using the particle capturing region. In a case where the confirmation is performed mechanically, the confirmation may be performed by the analysis unit 3 and/or the control unit 7 included in the microscopic system 100 according to the present technology.

In the confirmation, the order of discharge of the plurality of particles having distinguishment information can be referred to. For example, when the order of the pieces of distinguishment information of particles discharged by the discharge is different from the order of the pieces of distinguishment information of particles acquired by the distinguishment information acquiring unit for a particle group containing a predetermined number of continuous particles, the particle group can be discarded. For example, it is assumed that, in the discharge, a particle having red fluorescence, a particle having blue fluorescence, and a particle having red fluorescence are discharged in this order, and then the distinguishment information acquiring unit acquires distinguishment information of red, red, and blue. In this case, the confirmation unit compares the order of discharge in the discharge with the order of fluorescence acquired by the distinguishment information acquiring unit, and can confirm that these orders are different from each other. As a result, the confirmation unit can confirm that a target particle has not been acquired. Furthermore, it is also assumed that three particles having red fluorescence are continuously discharged in the discharge, and then the distinguishment information acquiring unit acquires distinguishment information of red, blue, and red. In this case, the confirmation unit compares the order of discharge in the discharge with the order of fluorescence acquired by the distinguishment information acquiring unit, and confirms that these orders are different from each other. As a result, the confirmation unit can confirm that a target particle has not been acquired. In a case where it is confirmed that the particles in the acquired particle group are not target particles, the acquired particles can be discarded.

Furthermore, the confirmation unit can compare the distinguishment information acquired by the distinguishment information acquiring unit with the distinguishment information acquired by the association unit. As a result of the comparison, in a case where these pieces of distinguishment information coincide with each other, it can be confirmed that the particle for which the distinguishment information is acquired in the distinguishment information acquiring unit is the same as the particle to be associated in the association unit. Since the distinguishment information and the position information are associated with each other for the particle to be associated in the association unit, it can be confirmed that the particle for which the distinguishment information is acquired in the distinguishment information acquiring unit has been captured in a well having the position information associated in the association unit. As a result of the comparison, in a case where these pieces of distinguishment information do not coincide with each other, it can be confirmed that the particle for which the distinguishment information is acquired in the distinguishment information acquiring unit is not the same as the particle to be associated in the association unit, and moreover, it can be confirmed that the particle for which the distinguishment information is acquired in the distinguishment information acquiring unit has not been captured in a well having the position information associated in the association unit.

In a case where the confirmation unit confirms that a particle to be extracted has not been extracted, a particle collected in a well may be discarded. Alternatively, the particle may be discarded without being collected in a well.

(5-5) Others

Furthermore, the particle extracting unit 5 can also be constituted by a conventionally known flow cytometer and the like. Specifically, a sample liquid containing a target particle is caused to flow into a flow cell, the sample liquid is irradiated with laser light, and measurement target light generated from the particle is detected. Examples of the measurement target light include scattered light such as forward scattered light, side scattered light, Rayleigh scattering, or Mie scattering, and fluorescence. The detected measurement target light is converted into an electric signal, and a particle extracting operation is performed on the basis of the electric signal.

Conventionally, a problem that original position information is lost occurs when all cells are collected from a cell array on a two-dimensional surface. However, in the present technology, a target particle is labeled using the dye, and even if extracting is performed by the particle extracting unit 5, original position information in the particle capturing unit 1 can be confirmed on the basis of the dye. Furthermore, by repeatedly performing primary labeling for dyeing a target particle with the particle labeling reagent and secondary labeling for dyeing the target particle that has obtained the primary labeling with a dye-labeled biotin-binding protein, and labeling a plurality of the target particles with different dyes, position information of each of the target particles can also be grasped.

(6) Observation Unit 6

The microscopic system 100 according to the present technology may include the observation unit 6 as necessary. The observation unit 6 observes a particle captured in a well. By observing a particle captured in a well, information such as the shape, structure, color, and the like of the particle, and the wavelength, intensity, and the like of light such as fluorescence generated from the particle can be obtained. Specifically, the observation unit 6 can be constituted by a microscope, a photodetector, or the like. The microscope is preferably an inverted microscope. Furthermore, the microscope is preferably an optical microscope. That is, in the microscopic system 100 according to the present technology, an inverted optical microscope is preferably used as the observation unit 6.

Furthermore, the observation unit 6 can include various light sources, various lenses, various filters, various mirrors, and the like.

(7) Control Unit 7

The microscopic system 100 according to the present technology may include the control unit 7 as necessary. The control unit 7 can control each unit included in the microscopic system 100, such as supply control of a particle and a fluid to the particle capturing unit 1, discharge control of a particle and a fluid from the particle capturing unit 1, control of image acquisition conditions in the image acquiring unit 2, control of irradiation conditions in the light irradiation unit 4, control of particle extracting conditions in the particle extracting unit 5, control of observation conditions in the observation unit 6, and control of analysis conditions in the analysis unit 3.

Note that, in the microscopic system 100 according to the present technology, the control unit 7 is not essential, and each unit can be controlled using an external control device and the like. Furthermore, the control unit 7 may be connected to each unit of the microscopic system 100 via a network.

(8) Storage Unit 8

The microscopic system 100 according to the present technology may include the storage unit 8 that stores various types of information as necessary. The storage unit 8 can store various data, conditions, and the like obtained in each unit of the microscopic system 100, such as information data regarding a particle captured state in the particle capturing unit 1, image data acquired by the image acquiring unit 2, data of a particle extracted by the particle extracting unit 5, observation data acquired by the observation unit 6, analysis data analyzed by the analysis unit 3, and control data in the control unit 7.

Note that, in the microscopic system 100 according to the present technology, the storage unit 8 is not essential, and various types of information can be stored using an external storage device and the like. As the storage unit 8, for example, a hard disk can be used. Furthermore, the storage unit 8 may be connected to each unit of the microscopic system 100 via a network.

(9) Display Unit 9

The microscopic system 100 according to the present technology may include the display unit 9 that displays various types of information as necessary. The display unit 9 can display various data, conditions, and the like obtained in each unit of the microscopic system 100, such as information data regarding a particle captured state in the particle capturing unit 1, observation data acquired by the observation unit 6, analysis data analyzed by the analysis unit 3, and control data in the control unit 7.

Note that, in the microscopic system 100 according to the present technology, the display unit 9 is not essential, and various types of information can be displayed using an external display device and the like. As the display unit 9, for example, a display or a printer can be used. Furthermore, the display unit 9 may be connected to each unit of the microscopic system 100 via a network.

### EXAMPLES

Hereinafter, the present technology will be described in more detail on the basis of Examples.

Note that Examples described below exemplify representative Examples of the present invention, and the scope of the present technology is not narrowly interpreted by Examples.

### Example 1

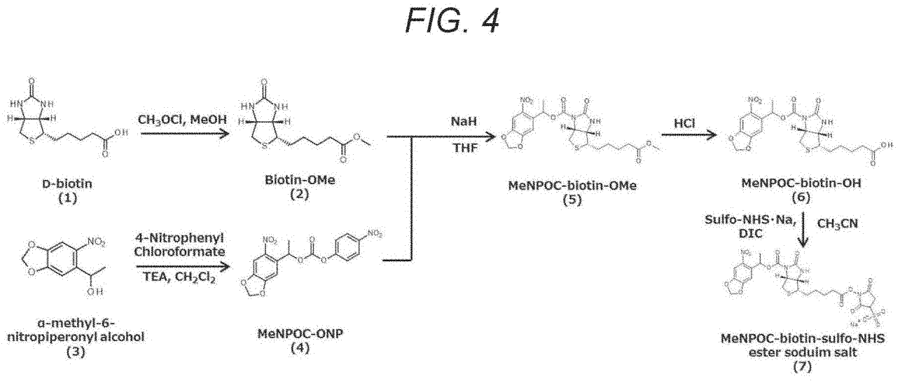

In the present Example 1, a MeNPOC-biotin-sulfo-NHS ester sodium salt was synthesized. The present synthetic scheme is illustrated in FIG. 4.

[Synthesis of Biotin-OMe (2)]

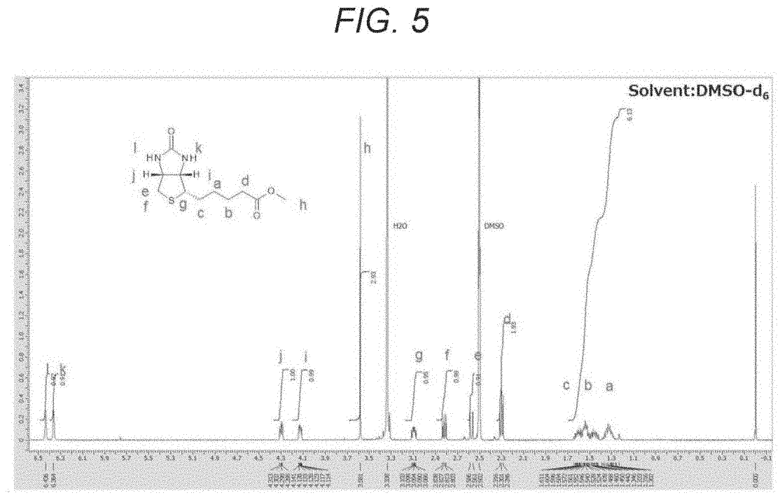

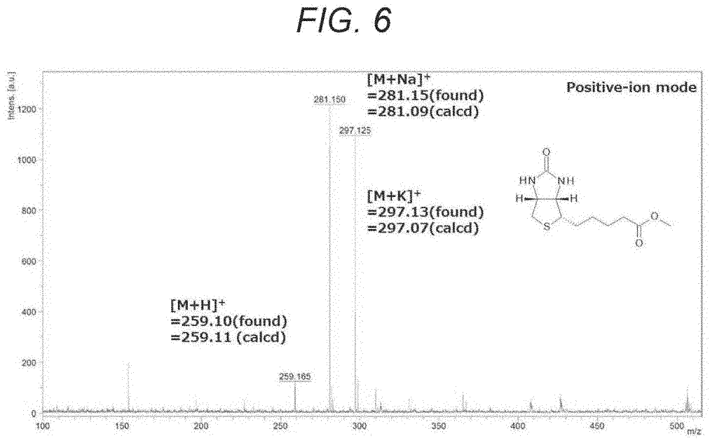

The inside of a 50 mL two-necked eggplant flask was purged with nitrogen, and then 13.3 mL of MeOH (dehydrated) was injected thereinto and ice-cooled. Next, 533 μL (7.5 mmol) of acetyl chloride was added dropwise one drop at a time to the eggplant flask. Ice water was removed, and the mixture was stirred at room temperature for five minutes. Thereafter, 979 mg (4.0 mmol) of D-biotin (1) suspended in 13.3 mL of MeOH (dehydrated) was added to the eggplant flask using a glass Pasteur. The mixture was stirred at room temperature for one hour and 30 minutes under nitrogen gas injection, and transferred to a 100 mL eggplant flask. The solvent was distilled off under reduced pressure at 40° C. Subsequently, a liquid separation operation was performed. The residue was transferred to a 200 mL separatory funnel, and 50 mL of 5% (v/v) MeOH/CH₂Cl₂ and 50 mL of saturated NaHCO₃/H₂O (>9.6 g/100 mL H₂O at 20° C.) were added thereto, stirred, and allowed to stand until the mixture was split into two layers (about 10 minutes). The lower layer (CH₂Cl₂ layer) was collected in a 100 mL conical beaker. 50 mL of 5% (v/v) MeOH/CH₂Cl₂ was added to the remaining aqueous layer, and the mixture was stirred again and allowed to stand. The lower layer (CH₂Cl₂ layer) was collected. Na₂SO₄ was added to the collected liquid, and the mixture was stirred for 30 minutes and filtered to remove residual moisture. The solvent was distilled off under reduced pressure at 40° C. and vacuum-dried in a desiccator for one hour to obtain a white dry solid. The product was confirmed by ¹H-NMR measurement and TOF MS (see FIGS. 5 and 6). The collected amount was 988 mg, and the yield was 95.7%.

[Synthesis of MeNPOC-ONP (4)]

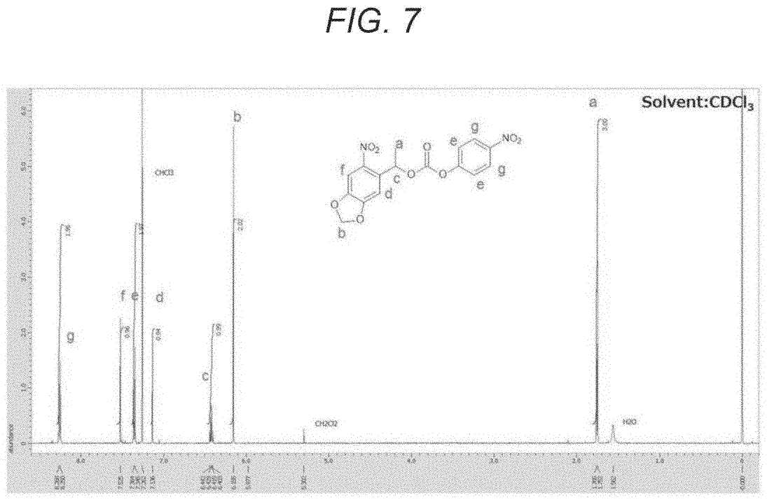

To 50 mL of a two-necked eggplant flask, 211 mg (1.0 mmol) of α-methyl-6 nitropiperonyl alcohol (3) and 403 mg (2.0 mmol) of 4-nitrophenyl chloroformate were added, and the inside of the two-necked eggplant flask was purged with nitrogen on ice. 10 mL of CH₂Cl₂ (dehydrated) was added thereto, and the mixture was stirred on ice for 15 minutes. 550 μL (4.0 mmol) of TEA was added thereto, and the mixture was further stirred on ice for 15 minutes. Subsequently, the mixture was stirred at room temperature overnight (14 hours). The reaction solution was purified by liquid chromatography (elution solvent:hexane:CH₂Cl₂=20:80 (w/w)). The extracted sample was collected in a 100 mL eggplant flask, and the solvent was distilled off under reduced pressure. The residue was vacuum-dried in a desiccator overnight to obtain a white solid. The product was confirmed by ¹H-NMR measurement (see FIG. 7). The collected amount was 340 mg, and the yield was 90.4%.

[Synthesis of MeNPOC-Biotin-OMe (5)]

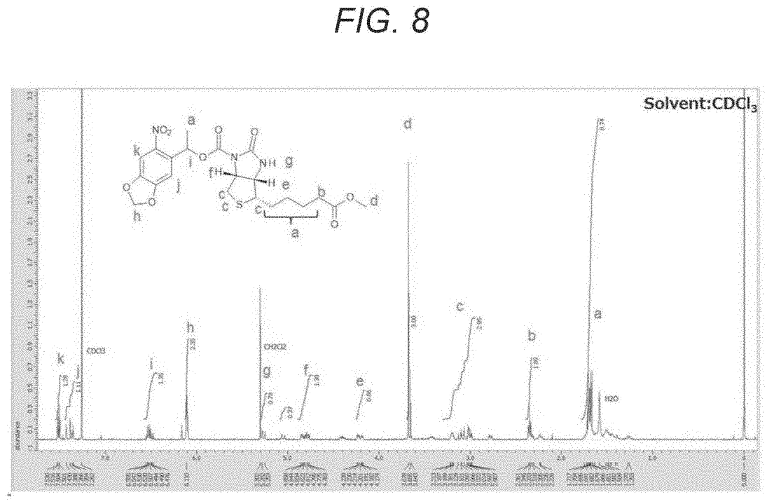

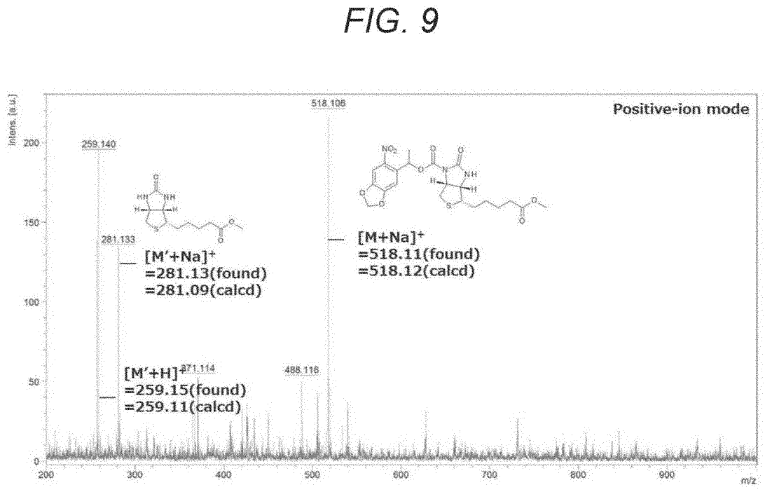

Into 50 mL of a two-necked eggplant flask, 258.1 mg (1.0 mmol) of biotin-OMe (2) and 188 mg (0.5 mmol) of MeNPOC-ONP (4) were put, and the inside of the two-necked eggplant flask was purged with nitrogen on ice. 10 mL of dehydrated THF was added thereto, and the mixture was stirred on ice for 15 minutes. Next, 42 mg (1.05 mmol) of NaH was added thereto, and the mixture was stirred on ice for 30 minutes and then further stirred at room temperature for six hours. The solution was ice-cooled for 15 minutes, and then the residual NaH was quenched with 120 μL (2.1 mmol) of acetic acid. The reaction solution was filtered, and the filtrate was collected in a 100 mL eggplant flask. The solvent was distilled off under reduced pressure at 30° C., and vacuum-dried in a desiccator for one hour and 30 minutes to obtain a yellow viscous solid. The solid was redissolved in 5 mL of 2% (v/v) MeOH/CH₂Cl₂ and purified by liquid chromatography (elution solvent:MeOH:CH₂Cl₂=1:99 (w/w)). Elution conditions are illustrated below. The extracted sample was collected in a 100 mL eggplant flask, and the solvent was distilled off under reduced pressure. The residue was vacuum-dried in a desiccator overnight. The product was confirmed by ¹H-NMR measurement and TOF MS (see FIGS. 8 and 9). The collected amount was 120 mg.

[Synthesis of MeNPOC-Biotin-OH (6)]

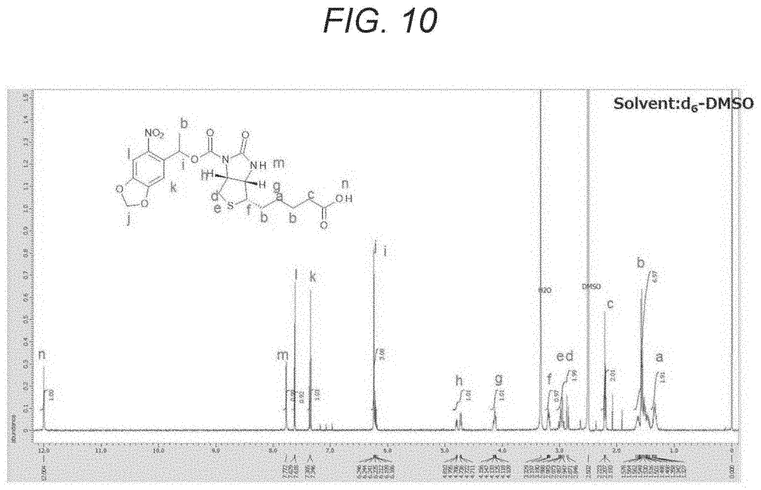

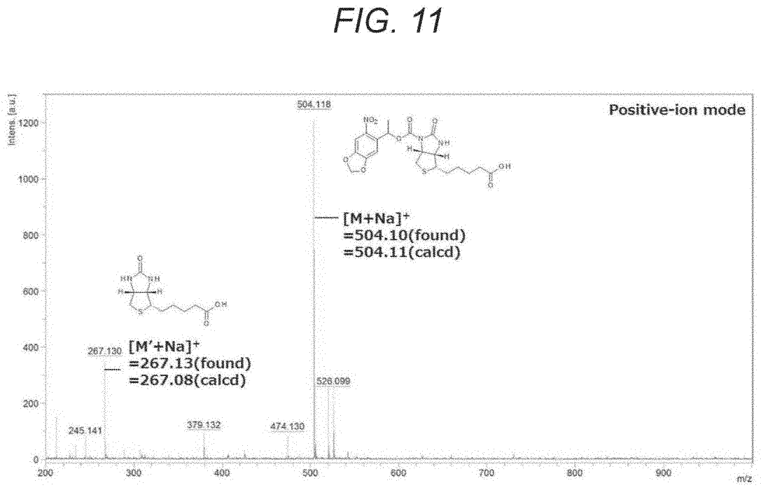

About 118 mg of the collected sample containing MeNPOC-biotin-OMe was dissolved in 10 mL of 0.5 M HCl/H₂O/50% (v/v) THF and transferred to a 50 mL three-necked flask. The mixture was stirred at 45° C. for 23 hours in an oil bath while nitrogen gas was fed to the three-necked flask. THF was distilled off under reduced pressure. Thereafter, 10 mL of ultrapure water was added thereto, and the mixture was lyophilized overnight (16 hours). Subsequently, the sample was redissolved in 2.5 mL of 50% (v/v) CH₃CN/H₂O and purified by liquid chromatography. The extracted sample was collected in a 100 mL eggplant flask, and the solvent was distilled off under reduced pressure. A yellow-white solid was obtained by the lyophilization. The product was confirmed by ¹H-NMR measurement and MALDI-TOF MS (see FIGS. 10 and 11). The collected amount was 65 mg. The yield was not calculated because the sample before hydrolysis was a mixture, but the yield was 27% by conversion from MeNPOC-ONP (4).

[Synthesis of MeNPOC-Biotin-Sulfo-NHS Ester Sodium Salt (7)]

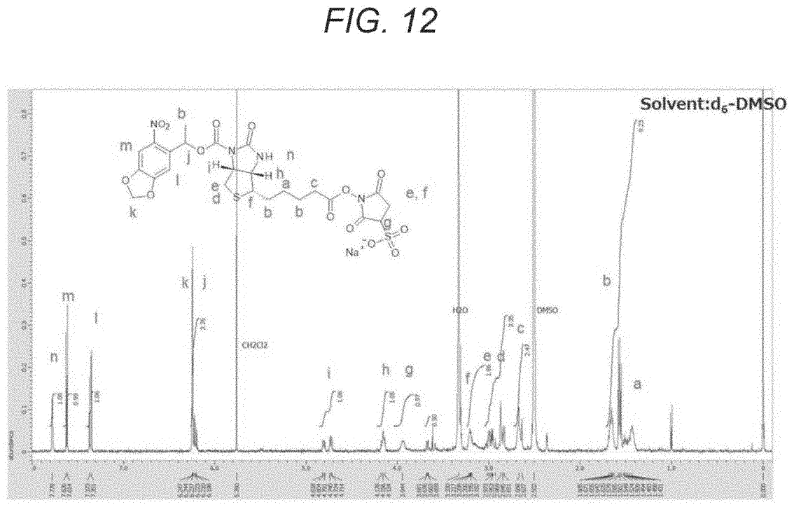

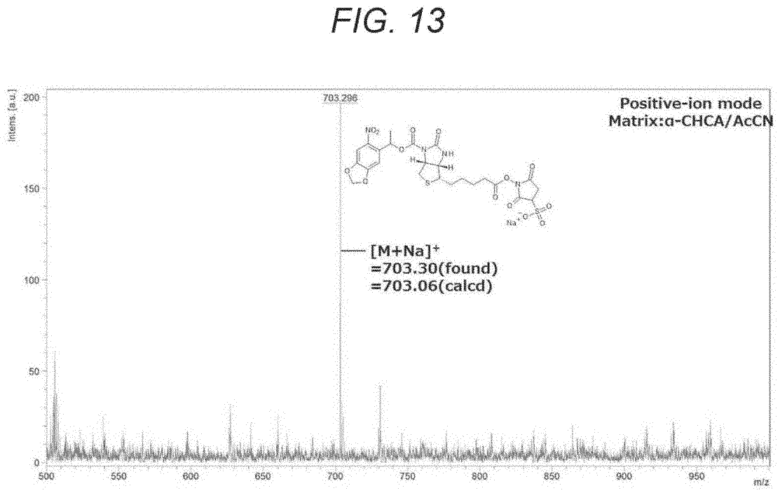

MeNPOC-biotin-OH (30 mg, 0.05 mmol) was stirred in 10 mL of dehydrated acetonitrile (AcCN), and 23.3 μL (0.15 mmol) of DIC and 32.6 mg (0.15 mmol) of N-hydroxysulfosuccinimide-Na (sulfo-NHS) were added thereto. The mixture was stirred at room temperature for 24 hours. Excess sulfo-NHS was removed by filtration, and then the solvent was distilled off under reduced pressure. 10 mL of CH₂Cl₂ was added thereto, and the mixture was allowed to stand. The precipitate was collected by filtration. The sample was vacuum-dried, and then the product was confirmed by ¹H-NMR measurement and MALDI-TOF MS (see FIGS. 12 and 13). The collected amount was 9.3 mg, and the yield was 27%.

### Example 2

In the present Example 2, photodegradation characteristics of MeNPOC biotin-OH were evaluated by ¹H-NMR.

<Evaluation Method>

MeNPOC-biotin-OH was dissolved in DMSO-d₆, and the concentration was adjusted to 0.566 mM. 700 μL of the solution was injected into each of two NMR sample tubes, and the NMR sample tubes were used for UV irradiation and non-UV irradiation, respectively. A UV irradiation distance was 5 cm (light intensity at 5 cm from a tip of an LED head was about 17 mW/cm²). The same sample was repeatedly subjected to UV irradiation and ¹H-NMR measurement. The photodegradation characteristics of the synthesized MeNPOC-biotin-OH were evaluated by ¹H-NMR. MeNPOC-biotin-OH was dissolved in DMSO-d₆, and then the solution was irradiated with UV (λₚ=365 nm) for a certain period of time.

<Evaluation Result>

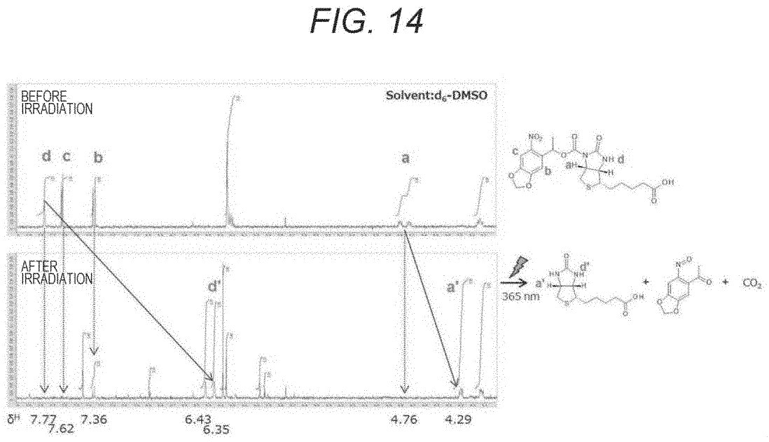

FIG. 14 illustrates 1H-NMR spectra before and after UV irradiation. Peaks a, b, c, and d decreased, and new peaks a′ and d′ appeared. a′ coincides with a chemical shift of a proton at position 9 on the thiophene ring of D-biotin, and d′ coincides with a chemical shift of an amine proton at position 3′ on the ureido ring thereof. It is considered that D-biotin is generated by photodegradation.

### Example 3

In the present Example 3, the amount of D-biotin generated by photodegradation by a HABA method was quantitatively evaluated.

<Evaluation Method>

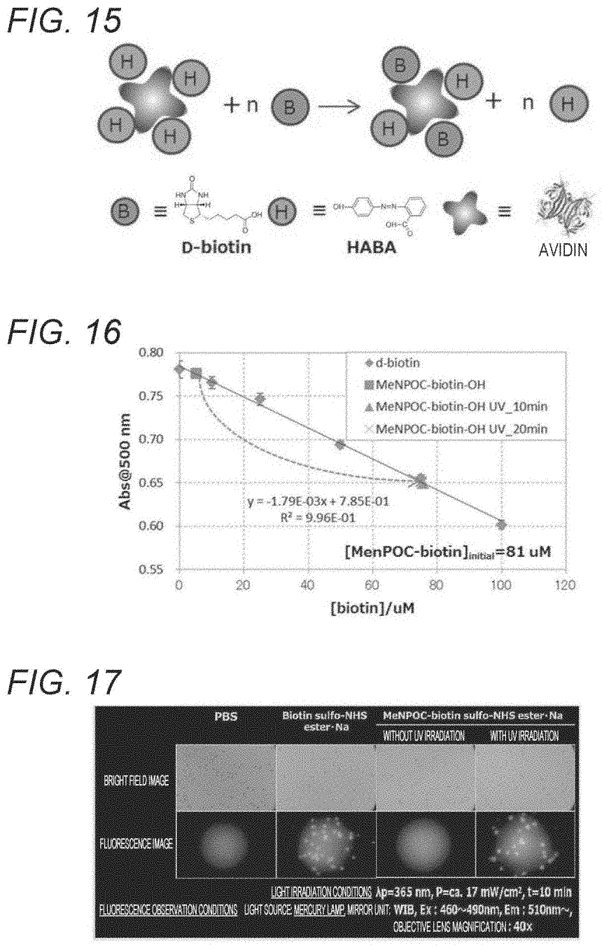

The generation amounts of D-biotin before and after MeNPOC-biotin-OH was irradiated with UV were evaluated by a HABA method (Green, NM., Methods Enzymol, 18-A, 418 (1970)) which is a D-biotin colorimetric method. First, a calibration curve was prepared using D-biotin. 4.4 mg of D-biotin was dissolved in 2.2 mL of DMSO, and 0.5 mL of the solution was mixed with 4.5 mL of 0.01 M PBS (containing no K) to prepare 0.82 mM D-biotin/10% (v/v) DMSO/PBS (containing no K). The concentration was further adjusted to 100 μM, 75 μM, 50 μM, 25 μM, and 10 μM with 10% (v/v) DMSO/PBS (containing no K). 14.5 mg (6.0×10⁻² mmol) of HABA was dissolved in 518 μL of DMSO. 4.8 mg (72.7 mmol) of Avidin, 9.504 mL of PBS (containing no K), and 96 μL of HABA/10% (v/v) DMSO were mixed ([avidin (monomer)]=30.3 μM, [HABA]=1.16 mM). 630 μL of HABA/avidin/DMSO/PBS (containing no K) and 70 μL of D-biotin/10% (v/v) DMSO/PBS (containing no K) were mixed, and then the mixture was allowed to stand for five minutes or more. The solution was added dropwise at 200 μL/well to a Pst 96 well plate (cat. #2-8085-02, Asone) so as to be added to three wells for each concentration, and absorbance (500 nm) was measured. As UV irradiation conditions, 100 μL of the solution was added dropwise to a COC film bottom 96 well plate (cat. #4680, Corning), and the solution was irradiated with UV light from a distance of 5 cm from the bottom surface. Irradiation time was 10 minutes or 20 minutes. The sample was collected from the well plate, mixed with HABA/avidin in a similar manner to D-biotin, and then absorbance was measured. A process in which D-biotin was generated by photodegradation of MeNPOC-biotin-OH and bound to avidin was indirectly evaluated by a HABA method (see FIG. 15). The HABA method is a biotin quantification method using a difference in affinity (dissociation constant) with respect to avidin between HABA and D-biotin (see Table 1 below). It is known that HABA has an absorption at 500 nm when HABA forms a complex with avidin, but decreases molar absorbance when HABA is released. Since HABA is replaced with D-biotin in the presence of D-biotin, the amount of D-biotin can be estimated from a change in absorbance at 500 nm.

TABLE 1
Dissociation constant of avidin complex

| | Dissociation constant |
|---|---|
| Complex | (unit: K_{d}/M) |
| HABA/avidin | 5.8 × 10⁻⁶ |
| D-biotin/avidin | 1 × 10⁻¹⁵ |

<Evaluation Result>

This time, when the amount of D-biotin generated by photodegradation from MeNPOC-biotin-OH was estimated using a HABA method, in the calibration curve using D-biotin, the amount of D-biotin was 5.1±1.6 μM before light irradiation and 75.7±1.8 μM 10 minutes after light irradiation in terms of D-biotin (see FIG. 16). The degradation concentration approximately coincided with the initial concentration (81 μM, in terms of a TFA salt), suggesting that almost the entire MeNPOC-biotin-OH was degraded.

### Example 4

In the present Example 4, dyeing of a Jurkat cell using a MeNPOC-biotin-sulfo-NHS ester sodium salt and a change in adsorption amount of fluorescently labeled streptavidin by UV irradiation were examined.

<Test Method>

1 mL of Jurkat cells 3.0×10⁶ cells/PBS (−), the Jurkat cells being human leukemia T cell lines, was prepared in a 1.5 mL Eppendorf tube, and centrifuged at 200×g for five minutes. The supernatant was removed, and 1 mL of a 50 μM MeNPOC-biotin-sulfo-NHS ester.Na/PBS solution was added thereto. The mixture was shielded from light by an aluminum foil, and allowed to stand at room temperature for 20 minutes. The mixture was centrifuged at 200×g for five minutes, and the supernatant was removed. The residue was resuspended in 1 mL of PBS (−) and centrifuged again at 200×g for five minutes, and the supernatant was removed. PBS (−) was added thereto, and the cell concentration was measured with a cell counter, and then the concentration was adjusted to 1.5×10⁵ cells/mL. The cells were added dropwise at 0.1 mL/well to a COC film bottom 96 well plate that had been soaked with 1% BSA overnight, and centrifuged at 120×g for one minute to precipitate the cells on a well bottom surface. Subsequently, the sample was irradiated with UV light from the well bottom surface side for 10 minutes using a UV-LED (LED 365-SPT, Optocode, λₚ=365 nm, about 17 mW/cm²). As a comparison, a sample not irradiated with UV light was allowed to stand for 10 minutes. The cell suspension was collected from the wells, and PBS was added thereto to adjust the volume to 0.8 mL. Thereafter, 200 μL of 50 μg/mL streptavidin conjugated with AlexaFluor 488/PBS was added thereto. The mixture was allowed to stand at room temperature for 10 minutes, and then centrifuged at 200×g for five minutes, and the supernatant was removed. The residue was resuspended in 1 mL of PBS (−) and centrifuged again at 200×g for five minutes, and the supernatant was removed again. The cells were resuspended in 0.5 mL of PBS and added dropwise at 0.1 mL/well to a COC film bottom 96 well plate. After centrifugation at 120×g for one minute, the cells were observed with a fluorescence microscope (IX-71, Olympus) (mirror unit: WIB, Ex: 460 to 490 nm/Em: 510 nm or more, objective lens: 40 times).

<Test Result>

It was observed that the fluorescence intensity changed depending on presence or absence of UV irradiation (see FIG. 17). This is considered to be because biotin was exposed by photodegradation, and fluorescently labeled streptavidin was easily adsorbed thereon.

### Example 5

In Example 5, an example of the particle dyeing method according to the present technology performed using the microscopic system 100 according to the present technology will be described.

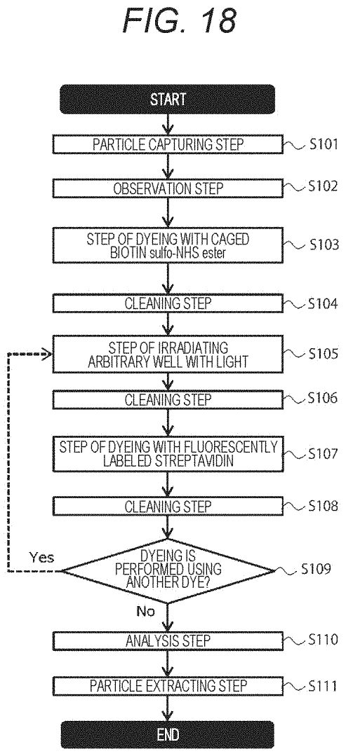

FIG. 18 is a flowchart illustrating an example of the particle dyeing method according to the present technology. First, particles such as cells are captured one by one by the particle capturing unit 1 (S101). Thereafter, the observation unit 6 observes a particles captured in a well (S102). Next, primary dyeing is performed with a MeNPOC-biotin-sulfo-NHS ester sodium salt (caged biotin sulfo-NHS ester) (S103). Thereafter, cleaning is performed (S104). Next, the light irradiation unit 4 irradiates an arbitrary well with light for uncaging (S105). Thereafter, cleaning is performed (S106), and dyeing is performed with fluorescently labeled streptavidin (S107). Next, cleaning is performed (S108).

Moreover, in a case where dyeing is performed using another dye (S109), the process returns to S105. A well different from the well previously irradiated with light is irradiated with light for uncaging, and the steps of S105 to S109 are repeated.

In a case where dyeing with a dye is completed (S109), the analysis unit 3 analyzes cells labeled with the dye (S110). Note that the analysis by the analysis unit 3 can be performed not only in S110 but also in S102. Thereafter, the particle extracting unit 5 extracts or selects a particle (S111).

Note that the present technology can have the following configurations.

(1)

A particle labeling reagent containing a compound represented by the above general formula (I-1) or (I-2). (In the above general formula (I-1), p represents an integer of 1 to 3.

In the above general formula (I-1), M represents a hydrogen atom or a mono- to tri-valent metal atom.

In the above general formula (I-1), L¹ represents a single bond or a (p+1)-valent group.

In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.

In the above general formula (I-2), L⁴ represents a monovalent group.)

(2)

The particle labeling reagent according to (1), in which L² and/or L³ in the general formulas (I-1) and (I-2) is a monovalent group containing a 2-nitrobenzyl derivative.

(3)

The particle labeling reagent according to (2), in which the monovalent group containing a 2-nitrobenzyl derivative is a monovalent group represented by any one of the above general formulas (II-1) to (II-3).

(In the above general formulas (II-1) to (II-3), R¹ and R⁶ each represent a hydrogen atom or a monovalent group. R¹ and R⁶ may be the same or different.

In the above general formulas (II-1) to (II-3), R², R³, R⁴, and R⁵ each independently represent a hydrogen atom or a monovalent group, or represent a ring structure formed by binding R², R³, R⁴, and R⁵ to each other. R², R³, R⁴, and R⁵ may be the same or different.

In the above general formulas (II-1) to (II-3), * represents a bond.)

(4)

The particle labeling reagent according to (3), in which any one or more of the group consisting of R², R³, R⁴, and R⁵ in the general formulas (II-1) to (II-3) each represent a monovalent group containing a polyethylene glycol chain.

(5)

The particle labeling reagent according to any one of (1) to (4), in which L¹ in the general formula (I-1) represents a (p+1)-valent group containing a succinimide ring.

(6)

The particle labeling reagent according to any one of (1) to (5), in which L¹ in the general formula (I-1) represents a (p+1)-valent group containing a polyethylene glycol chain.

(7)

The particle distinguishing and labeling reagent according to any one of (1) to (4), in which L⁴ in the general formula (I-2) represents a monovalent lipid-soluble functional group.

(8)

The particle distinguishing and labeling reagent according to any one of (1) to (4) and (7), in which L⁴ in the general formula (I-2) represents a monovalent group containing a polyethylene glycol chain.

(9)

The particle distinguishing and labeling reagent according to any one of (1) to (4), in which L⁴ in the general formula (I-2) represents a monovalent cationic functional group.

(10)

A particle dyeing method including:

a primary labeling step of dyeing a target particle with a particle labeling reagent containing a compound represented by the above general formula (I-1) or (I-2) and irradiating the dyed target particle with light; and

a secondary labeling step of dyeing the target particle that has been subjected to the primary labeling step with a dye-labeled biotin-binding protein.

(In the above general formula (I-1), p represents an integer of 1 to 3.

In the above general formula (I-1), M represents a hydrogen atom or a mono- to tri-valent metal atom.

In the above general formula (I-1), L¹ represents a single bond or a (p+1)-valent group.

In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.

In the above general formula (I-2), L⁴ represents a monovalent group.)

(11)

The particle dyeing method according to (10), in which the primary labeling step further includes a binding enabling step in which the photodegradable protecting group is degraded by light irradiation and biotin becomes capable of binding to a biotin-binding protein.

(12)

The particle dyeing method according to (10) or (11), in which by repeatedly performing the primary labeling step and the secondary labeling step, biotin-binding proteins in the different secondary labeling steps are labeled with different dyes.

(13)

A microscopic system including:

a particle capturing unit that captures a target particle in a well in a particle capturing region;

an image acquiring unit that acquires an image of the captured target particle; and

an analysis unit that analyzes the image of the target particle acquired by the image acquiring unit, in which the target particle analyzed by the analysis unit is dyed with a particle labeling reagent containing a compound represented by the above general formula (I-1) or (I-2).

(In the above general formula (I-1), p represents an integer of 1 to 3.

In the above general formula (I-1), M represents a hydrogen atom or a mono- to tri-valent metal atom.

In the above general formula (I-1), L¹ represents a single bond or a (p+1)-valent group.

In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.

In the above general formula (I-2), L⁴ represents a monovalent group.)

(14)

The microscopic system according to (13), further including a light irradiation unit that emits light, in which

the dyed target particle is primarily labeled by being irradiated with light by the light irradiation unit, and the primarily labeled target particle becomes capable of binding to a dye-labeled biotin-binding protein.

(15)

The microscopic system according to (13) or (14), further including a particle extracting unit that extracts a target particle, in which

the particle extracting unit extracts the target particle binding to the dye-labeled biotin-binding protein.

### REFERENCE SIGNS LIST

100 Microscopic system
1 Particle capturing unit
2 Image acquiring unit
3 Analysis unit
4 Light irradiation unit
5 Particle extracting unit
6 Observation unit
7 Control unit
8 Storage unit
9 Display unit

1. A particle labeling reagent comprising a compound represented by the following general formula (I-1) or (I-2). 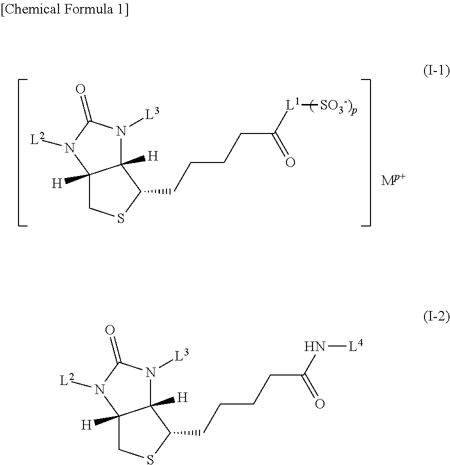
(In the above general formula (I-1), p represents an integer of 1 to 3.
In the above general formula (I-1), M represents a hydrogen atom or a mono- to tri-valent metal atom.
In the above general formula (I-1), L¹ represents a single bond or a (p+1)-valent group.
In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.
In the above general formula (I-2), L⁴ represents a monovalent group.)

2. The particle labeling reagent according to claim 1, wherein L² and/or L³ in the general formulas (I-1) and (I-2) is a monovalent group containing a 2-nitrobenzyl derivative.

3. The particle labeling reagent according to claim 2, wherein the monovalent group containing a 2-nitrobenzyl derivative is a monovalent group represented by any one of the following general formulas (II-1) to (II-3). 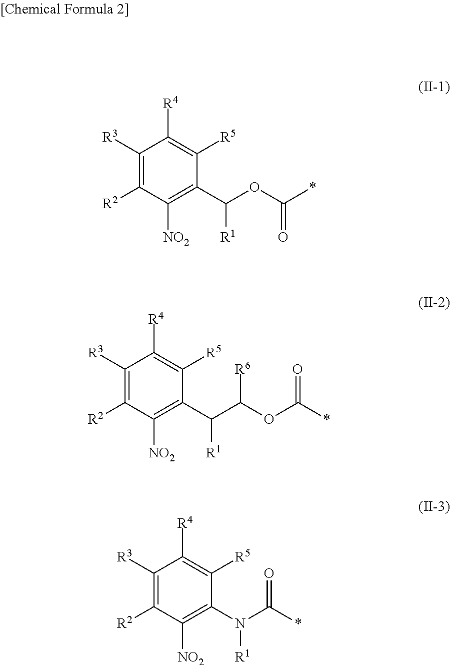
(In the above general formulas (II-1) to (II-3), R¹ and R⁶ each represent a hydrogen atom or a monovalent group. R¹ and R⁶ may be the same or different.
In the above general formulas (II-1) to (II-3), R², R³, R⁴, and R⁵ each independently represent a hydrogen atom or a monovalent group, or represent a ring structure formed by binding R², R³, R⁴, and R⁵ to each other. R², R³, R⁴, and R⁵ may be the same or different.
In the above general formulas (II-1) to (II-3), * represents a bond.)

4. The particle labeling reagent according to claim 3, wherein any one or more of the group consisting of R², R³, R⁴, and R⁵ in the general formulas (II-1) to (II-3) each represent a monovalent group containing a polyethylene glycol chain.

5. The particle labeling reagent according to claim 1, wherein L¹ in the general formula (I-1) represents a (p+1)-valent group containing a succinimide ring.

6. The particle labeling reagent according to claim 1, wherein L¹ in the general formula (I-1) represents a (p+1)-valent group containing a polyethylene glycol chain.

7. The particle distinguishing and labeling reagent according to claim 1, wherein L⁴ in the general formula (I-2) represents a monovalent lipid-soluble functional group.

8. The particle distinguishing and labeling reagent according to claim 1, wherein L⁴ in the general formula (I-2) represents a monovalent group containing a polyethylene glycol chain.

9. The particle distinguishing and labeling reagent according to claim 1, wherein L⁴ in the general formula (I-2) represents a monovalent cationic functional group.

10. A particle dyeing method comprising:
a primary labeling step of dyeing a target particle with a particle labeling reagent containing a compound represented by the following general formula (I-1) or (I-2) and irradiating the dyed target particle with light; and
a secondary labeling step of dyeing the target particle that has been subjected to the primary labeling step with a dye-labeled biotin-binding protein. 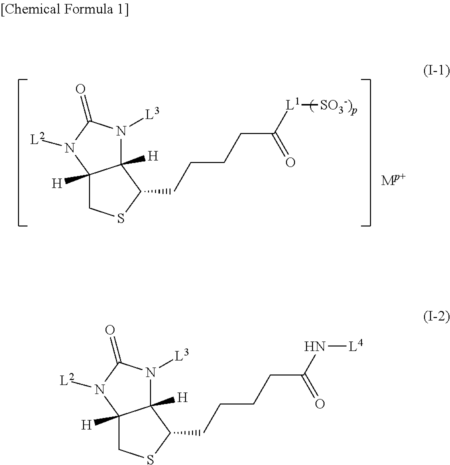
(In the above general formula (I-1), p represents an integer of 1 to 3.
In the above general formula (I-1), M represents a hydrogen atom or a mono- to tri-valent metal atom.
In the above general formula (I-1), L¹ represents a single bond or a (p+1)-valent group.
In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.
In the above general formula (I-2), L⁴ represents a monovalent group.)

11. The particle dyeing method according to claim 10, wherein the primary labeling step further includes a binding enabling step in which the photodegradable protecting group is degraded by light irradiation and biotin becomes capable of binding to a biotin-binding protein.

12. The particle dyeing method according to claim 10, wherein by repeatedly performing the primary labeling step and the secondary labeling step, biotin-binding proteins in the different secondary labeling steps are labeled with different dyes.

13. A microscopic system comprising:
a particle capturing unit that captures a target particle in a well in a particle capturing region;
an image acquiring unit that acquires an image of the captured target particle; and
an analysis unit that analyzes the image of the target particle acquired by the image acquiring unit, wherein
the target particle analyzed by the analysis unit is dyed with a particle labeling reagent containing a compound represented by the following general formula (I-1) or (I-2). 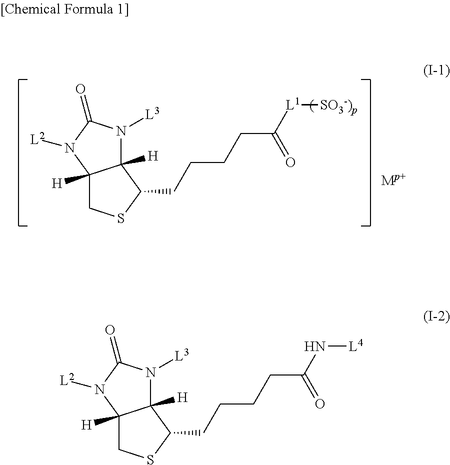
(In the above general formula (I-1), p represents an integer of 1 to 3.
In the above general formula (I-1), M represents a hydrogen atom or a mono- to tri-valent metal atom.
In the above general formula (I-1), L¹ represents a single bond or a (p+1)-valent group.
In the above general formulas (I-1) and (I-2), L² and L³ each independently represent a hydrogen atom or a photodegradable protecting group, and L² and L³ may be the same or different. Provided that at least one of L² and L³ represents a photodegradable protecting group.
In the above general formula (I-2), L⁴ represents a monovalent group.)

14. The microscopic system according to claim 13, further comprising a light irradiation unit that emits light, wherein
the dyed target particle is primarily labeled by being irradiated with light by the light irradiation unit, and the primarily labeled target particle becomes capable of binding to a dye-labeled biotin-binding protein.

15. The microscopic system according to 13, further comprising a particle extracting unit that extracts a target particle, wherein
the particle extracting unit extracts the target particle binding to the dye-labeled biotin-binding protein.
